SEQUENCE LISTING (75 sequences)

SEQ ID NO: 1 - protein, 119 aa, synthetic construct
QVQLQQSGAELVRPGASVTLSCKASGYTFTAYEMHWVRQTPVNGLEWIGAIDPKTGVTAYNQKFKDKAIMTADKYSSTAYMDLRSLTSEDSAVYYCTSRAGGRGRFAYWGQGTLVTVSA

SEQ ID NO: 2 - protein, 5 aa, synthetic construct
AYEMH

SEQ ID NO: 3 - protein, 17 aa, synthetic construct
AIDPKTGVTAYNQKFKD

SEQ ID NO: 4 - protein, 10 aa, synthetic construct
RAGGRGRFAY

SEQ ID NO: 5 - protein, 112 aa, synthetic construct
DVVMTQTPLSLPVSLGDQASISCRSSQSLVHSSANTYLHWYLQKPGQSPKVLIYKVSNRFSGVPDRFSGSGSGTDFTLKISRVEAEDLGLYFCSQSTHVPFTFGGGTKLEIK

SEQ ID NO: 6 - protein, 16 aa, synthetic construct
RSSQSLVHSSANTYLH

SEQ ID NO: 7 - protein, 7 aa, synthetic construct
KVSNRFS

SEQ ID NO: 8 - protein, 9 aa, synthetic construct
SQSTHVPFT

SEQ ID NO: 9 - protein, 119 aa, synthetic construct
QVQLQQSGAELARPGASVTLSCKASGYTFTDYEMHWVKQTPVRGLEWIGAIDPETGATAYNQKFKGKATLTADKSSNTAYMELRRLTSEDSAVYYCKSRAGGRGRFAYWGQGTLVTVSA

SEQ ID NO: 10 - protein, 5 aa, synthetic construct
DYEMH

SEQ ID NO: 11 - protein, 17 aa, synthetic construct
AIDPETGATAYNQKFKG

SEQ ID NO: 12 - protein, 10 aa, synthetic construct
RAGGRGRFAY

SEQ ID NO: 13 - protein, 112 aa, synthetic construct
DVVMTQTPLSLPVRLGDQASISCRSSQSLVHSSGNTYLHWYLQKSGQSPKLLIYKVSNRFSGVPDRFSGSGSGTDFTLKISRVEAEDLGVYFCSQTTHVPYTFGGGTKLEIK

SEQ ID NO: 14 - protein, 16 aa, synthetic construct
RSSQSLVHSSGNTYLH

SEQ ID NO: 15 - protein, 7 aa, synthetic construct
KVSNRFS

SEQ ID NO: 16 - protein, 9 aa, synthetic construct
SQTTHVPYT

SEQ ID NO: 17 - protein, 124 aa, synthetic construct
EVQLVETGGGLVQPKGSLKLSCAASGFTFNTYAMNWVRQTPEKGLEWVARIRTKSNNYATSYADSVKDRFTISRYDSQNMLYLQMNNLKTEDTAMYYCVRGGYGIRGDYAMDYWGQGTSVTVSS

SEQ ID NO: 18 - protein, 5 aa, synthetic construct
TYAMN

SEQ ID NO: 19 - protein, 19 aa, synthetic construct
RIRTKSNNYATSYADSVKD

SEQ ID NO: 20 - protein, 13 aa, synthetic construct
GGYGIRGDYAMDY

SEQ ID NO: 21 - protein, 112 aa, synthetic construct
DIVMTQAAPSVPVTPGESVSISCRSTKSLLHSNGNTYLYWFLQRPGQSPQLLIYRMSNLASGVPDRFSGSGSGTTFTLRISRVEAEDVGVYYCMQHLEYPFTFGSGTKLEIK

SEQ ID NO: 22 - protein, 16 aa, synthetic construct
RSTKSLLHSNGNTYLY

SEQ ID NO: 23 - protein, 7 aa, synthetic construct
RMSNLAS

SEQ ID NO: 24 - protein, 9 aa, synthetic construct
MQHLEYPFT

SEQ ID NO: 25 - protein, 124 aa, synthetic construct
EVQLVETGGGLVQPKGSLKLSCAASGFTFNTNAMNWVRQAPGKGLEWVGRIRTKSNNYATYYADSVRDRFTISRYDSQGMLYLQMNNLKTEDTAMYYCVRGGYGIRGDYTMDYWGQGTSVTVSS

SEQ ID NO: 26 - protein, 5 aa, synthetic construct
TNAMN

SEQ ID NO: 27 - protein, 19 aa, synthetic construct
RIRTKSNNYATYYADSVRD

SEQ ID NO: 28 - protein, 13 aa, synthetic construct
GGYGIRGDYTMDY

SEQ ID NO: 29 - protein, 112 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 30 - protein, 16 aa, synthetic construct
RSSKSLLHSNGNTYLY

SEQ ID NO: 31 - protein, 7 aa, synthetic construct
RMSNLAS

SEQ ID NO: 32 - protein, 9 aa, synthetic construct
MQHLEYPFT

SEQ ID NO: 33 - protein, 119 aa, synthetic construct
QVQLVQSGAEVKKPGASVKVSCKASGYTFTDYEMHWVRQAPGQGLEWMGAIDPETGATAYNQKFKGRVTSTRDTSISTAYMELSRLRSDDTVVYYCARRAGGRGRFAYWGQGTLVTVSS

SEQ ID NO: 34 - protein, 119 aa, synthetic construct
QVQLVQSGAEVKKPGASVKVSCKASGYTFTDYEMHWVRQAPGQGLEWMGAIDPETGATAYNQKFKGRVTLTADTSISTAYMELSRLRSDDTVVYYCASRAGGRGRFAYWGQGTLVTVSS

SEQ ID NO: 35 - protein, 119 aa, synthetic construct
QVQLVQSGAEVKKPGASVKVSCKASGYTFTDYEMHWVRQAPGQGLEWMGAIDPETGATAYNQKFKGRVTLTADTSISTAYMELSRLRSDDTVVYYCKSRAGGRGRFAYWGQGTLVTVSS

SEQ ID NO: 36 - protein, 119 aa, synthetic construct
QVQLVQSGAEVKKPGASVKVSCKASGYTFTDYEMHWVRQAPVRGLEWMGAIDPETGATAYNQKFKGRVTLTADTSISTAYMELSRLRSDDTVVYYCKSRAGGRGRFAYWGQGTLVTVSS

SEQ ID NO: 37 - protein, 112 aa, synthetic construct
EIVLTQSPGTLSLSPGERATLSCRSSQSLVHSSGNTYLHWYQQKPGQAPRLLIYKVSNRFSGIPDRFSGSGSGTDFTLTISRLEPEDFAVYYCSQTTHVPYTFGGGTKLELK

SEQ ID NO: 38 - protein, 112 aa, synthetic construct
EVVMTQSPGTLSLSPGERATLSCRSSQSLVHSSGNTYLHWYQQKPGQSPRLLIYKVSNRFSGIPDRFSGSGSGTDFTLTISRLEPEDFAVYFCSQTTHVPYTFGGGTKLELK

SEQ ID NO: 39 - protein, 112 aa, synthetic construct
DVVMTQSPLTLSLSPGERATLSCRSSQSLVHSSGNTYLHWYQQKPGQSPKLLIYKVSNRFSGIPDRFSGSGSGTDFTLTISRLEPEDFAVYFCSQTTHVPYTFGGGTKLELK

SEQ ID NO: 40 - protein, 124 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAVYYCARGGYGIRGDYTMDYWGQGTLVTVSS

SEQ ID NO: 41 - protein, 10 aa, synthetic construct
GFTFNTNAMN

SEQ ID NO: 42 - protein, 19 aa, synthetic construct
RIRTKSNNYATYYAESVRD

SEQ ID NO: 43 - protein, 13 aa, synthetic construct
GGYGIRGDYTMDY

SEQ ID NO: 44 - protein, 124 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGDYTMDYWGQGTLVTVSS

SEQ ID NO: 45 - protein, 10 aa, synthetic construct
GFTFNTNAMN

SEQ ID NO: 46 - protein, 19 aa, synthetic construct
RIRTKSNNYATYYAESVRD

SEQ ID NO: 47 - protein, 13 aa, synthetic construct
GGYGIRGDYTMDY

SEQ ID NO: 48 - protein, 124 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSS

SEQ ID NO: 49 - protein, 10 aa, synthetic construct
GFTFNTNAMN

SEQ ID NO: 50 - protein, 19 aa, synthetic construct
RIRTKSNNYATYYAESVRD

SEQ ID NO: 51 - protein, 13 aa, synthetic construct
GGYGIRGEYTMDY

SEQ ID NO: 52 - protein, 124 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRADYTMDYWGQGTLVTVSS

SEQ ID NO: 53 - protein, 10 aa, synthetic construct
GFTFNTNAMN

SEQ ID NO: 54 - protein, 19 aa, synthetic construct
RIRTKSNNYATYYAESVRD

SEQ ID NO: 55 - protein, 13 aa, synthetic construct
GGYGIRADYTMDY

SEQ ID NO: 56 - protein, 112 aa, synthetic construct
EIVLTQSPGTLSLSPGERATLSCRSSKSLLHSNGNTYLYWYQQKPGQAPRLLIYRMSNLASGIPDRFSGSGSGTDFTLTISRLEPEDFAVYYCMQHLEYPFTFGQGTKLEIK

SEQ ID NO: 57 - protein, 16 aa, synthetic construct
RSSKSLLHSNGNTYLY

SEQ ID NO: 58 - protein, 7 aa, synthetic construct
RMSNLAS

SEQ ID NO: 59 - protein, 9 aa, synthetic construct
MQHLEYPFT

SEQ ID NO: 60 - protein, 112 aa, synthetic construct
EIVMTQSPGTLSLSPGERATLSCRSSKSLLHSNGNTYLYWFQQKPGQSPRLLIYRMSNLASGIPDRFSGSGSGTDFTLTISRLEPEDFAVYYCMQHLEYPFTFGQGTKLEIK

SEQ ID NO: 61 - protein, 16 aa, synthetic construct
RSSKSLLHSNGNTYLY

SEQ ID NO: 62 - protein, 7 aa, synthetic construct
RMSNLAS

SEQ ID NO: 63 - protein, 9 aa, synthetic construct
MQHLEYPFT

SEQ ID NO: 64 - protein, 112 aa, synthetic construct
DIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIK

SEQ ID NO: 65 - protein, 16 aa, synthetic construct
RSSKSLLHSNGNTYLY

SEQ ID NO: 66 - protein, 7 aa, synthetic construct
RMSNLAS

SEQ ID NO: 67 - protein, 9 aa, synthetic construct
MQHLEYPFT

SEQ ID NO: 68 - protein, 112 aa, synthetic construct
DIVMTQSPPTLSLSPGERATLSCRSSKSLLHSQGNTYLYWFQQKPGQSPQLLIYRMSNLASGIPDRFSGSGSGTDFTLTISRLEPEDFAVYYCMQHLEYPFTFGQGTKLEIK

SEQ ID NO: 69 - protein, 16 aa, synthetic construct
RSSKSLLHSQGNTYLY

SEQ ID NO: 70 - protein, 7 aa, synthetic construct
RMSNLAS

SEQ ID NO: 71 - protein, 9 aa, synthetic construct
MQHLEYPFT

SEQ ID NO: 72 - protein, 112 aa, synthetic construct
DIVMTQSPPTLSLSPGERATLSCRSSKSLLHSNANTYLYWFQQKPGQSPQLLIYRMSNLASGIPDRFSGSGSGTDFTLTISRLEPEDFAVYYCMQHLEYPFTFGQGTKLEIK

SEQ ID NO: 73 - protein, 16 aa, synthetic construct
RSSKSLLHSNANTYLY

SEQ ID NO: 74 - protein, 7 aa, synthetic construct
RMSNLAS

SEQ ID NO: 75 - protein, 9 aa, synthetic construct
MQHLEYPFT

(19) United States
(12) Patent Application Publication
(10) Pub. No.: US 2025/0243287 A1
(43) Pub. Date: Jul. 31, 2025

(54) ANTI-CCR8 ANTIBODY OR ANTIGEN-BINDING FRAGMENT THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Chunling QIN, Nanjing (CN); Xiaoling JIANG, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 18/854,189

(22) PCT Filed: Apr. 6, 2023
(86) PCT No.: PCT/CN2023/086391

§ 371 Date: Oct. 4, 2024

(30) Foreign Application Priority Data
Apr. 7, 2022 (WO) PCT/CN2022/085464

(51) Int. Cl. C07K 16/28; A61K 39/00; A61P 35/00

(52) U.S. Cl. CPC C07K 16/2866; A61P 35/00; A61K 2039/505; C07K 2317/24; C07K 2317/732; C07K 2317/92

(57) ABSTRACT

An anti-CCR8 antibody or antigen-binding fragment thereof. The anti-CCR8 antibody can recognize human CCR8 protein, block the signal pathway between CCR8 and CCL1/CCL18, and recognize cells expressing CCR8, and also has target specific ADCC activity on target cells expressing CCR8.

### CROSS-REFERENCES TO RELATED APPLICATIONS

This application is a national phase entry under 35 U.S.C § 371 of International Application No. PCT/CN2023/086391, filed on Apr. 6, 2023, which claims priority to International Application No. PCT/CN2022/085464, filed on Apr. 7, 2022, the entire contents of all of which are incorporated herein by reference.

### REFERENCE TO AN ELECTRONIC SEQUENCE LISTING

The contents of the electronic sequence listing (SH20240918; Size: 67.1 kilobytes; and Date of Creation: Sep. 18, 2024) is herein incorporated by reference in its entirety.

### TECHNICAL FIELD

The present invention belongs to the field of tumor immunotherapy and molecular immunology and specifically relates to an anti-CCR8 antibody or antigen-binding fragment thereof.

### BACKGROUND

Chemokine receptors (Chemoreceptors) are some seven-transmembrane G protein-coupled receptors (GPCRs) expressed on the surface of some specific cells. Chemokine receptors bind to extracellular ligand chemokines, triggering a chemotactic response by calcium inward flow, thereby inducing cells to specific sites in the organism. The binding of CCR8 and its ligands CCL1/CCL18 positively feedback regulates the immunosuppressive function of the tumor microenvironment.

CCR8 (chemokine (C-C motif) receptor 8) is a chemokine receptor highly expressed in tumor-infiltrating Tregs, with a structure similar to that of G-protein-coupled receptors, and belongs to the group of seven-transmembrane proteins. CCR8 is specifically expressed on tumor infiltrating regulatory T cells (Tregs), but not significantly expressed on peripheral blood Tregs, regulating the immune suppressive function of the tumor microenvironment. CCR8 induces proliferation, migration and apoptosis resistance of cancer cells by activating the CCR8 receptor on cancer cells. In addition, CCL1 recruits Treg to the tumor ecological niche and leads to the conversion of CD4+ T cells into Treg.

There are four known receptors for CCR8: CCL1, CCL8, CCL16, and CCL18, of which the primary receptor, the chemokine CCL1, is upregulated in expression at sites of inflammation. CCL1 is able to recruit FOXp3+CCR8+ Treg cells to infiltrate into tumor tissues and exert immunosuppressive functions, while it can induce up-regulation of CCR8 expression on the surface of FOXp3+ Treg cells, induce Ca2+ mobility, and induces up-regulation of stat3-dependent expression of Foxp3, CD39, IL-10, and granzyme B, which then enhances the immunosuppressive activity of these tumor-infiltrating Treg cells. Thus, drugs targeting CCR8 can enhance anti-tumor immune effects by depleting tumor-infiltrating FOXp3+CCR8+ Treg cells, or by blocking the CCL1/CCR8 pathway.

### SUMMARY

The anti-CCR8 antibody developed in the present invention can specifically recognize human CCR8 protein, can block the signal pathway between CCR8 and CCL1/CCL18, can specifically recognize cells expressing CCR8, and has target specific ADCC activity on target cells expressing CCR8.

The technical solutions used in the present invention are as follows:
An anti-CCR8 antibody or antigen-binding fragment thereof, comprising a heavy chain variable region and a light chain variable region, wherein the heavy chain variable region comprises the heavy chain complementary determining regions HCDR1, HCDR2 and HCDR3, and the light chain variable region comprises the light chain complementary determining regions LCDR1, LCDR2 and LCDR3, wherein,
HCDR1 of the heavy chain variable region is selected from any amino acid sequence of any of SEQ ID NO: 2, 10, 18, 26, 41, 45, 49, 53, or a sequence having at least 80%, 85%, 90%, 91%, 92%, 93%, 94%, 95%, 96%, 97%, 98%, 99% or more identity to the amino acid sequence of any of SEQ ID NO: 2, 10, 18, 26, 41, 45, 49, 53, or an amino acid sequence having one or more (preferably 2 or 3) conserved amino acid mutations (preferably substitutions, insertions, or deletions) as compared to the amino acid sequence of any of SEQ ID NO: 2, 10, 18, 26, 41, 45, 49, 53;
(b) HCDR2 of the heavy chain variable region is selected from any amino acid sequence of any of SEQ ID NO: 3, 11, 19, 27, 42, 46, 50, 54, or a sequence having at least 80%, 85%, 90%, 91%, 92%, 93%, 94%, 95%, 96%, 97%, 98%, 99% or more identity to the amino acid sequence of any of SEQ ID NO: 3, 11, 19, 27, 42, 46, 50, 54, or an amino acid sequence having one or more (preferably 2 or 3) conserved amino acid mutations (preferably substitutions, insertions, or deletions) as compared to the amino acid sequence of any of SEQ ID NO: 3, 11, 19, 27, 42, 46, 50, 54;
(c) HCDR3 of the heavy chain variable region is selected from any amino acid sequence of any of SEQ ID NO: 4, 12, 20, 28, 43, 47, 51, 55, or a sequence having at least 80%, 85%, 90%, 91%, 92%, 93%, 94%, 95%, 96%, 97%, 98%, 99% or more identity to the amino acid sequence of any of SEQ ID NO: 4, 12, 20, 28, 43, 47, 51, 55, or an amino acid sequence having one or more (preferably 2 or 3) conserved amino acid mutations (preferably substitutions, insertions, or deletions) as compared to the amino acid sequence of any of SEQ ID NO: 4, 12, 20, 28, 43, 47, 51, 55;
(d) LCDR1 of the light chain variable region is selected from any amino acid sequence of SEQ ID NO: 6, 14, 22, 30, 57, 61, 65, 69, 73, or a sequence having at least 80%, 85%, 90%, 91%, 92%, 93%, 94%, 95%, 96%, 97%, 98%, 99% or more identity to any amino acid sequence of SEQ ID NO: 6, 14, 22, 30, 57, 61, 65, 69, 73, or an amino acid sequence having one or more (preferably 2 or 3) conserved amino acid mutations (preferably substitutions, insertions, or deletions) in comparison to any amino acid sequence of SEQ ID NO: 6, 14, 22, 30, 57, 61, 65, 69, 73;
(e) LCDR2 of the light chain variable region is selected from any amino acid sequence of SEQ ID NO: 7, 15, 23, 31, 58, 62, 66, 70, 74, or a sequence having at least 80%, 85%, 90%, 91%, 92%, 93%, 94%, 95%, 96%, 97%, 98%, 99% or more identity to any amino acid sequence of SEQ ID NO: 7, 15, 23, 31, 58, 62, 66, 70, 74, or an amino acid sequence having one or more (preferably 2 or 3) conserved amino acid mutations (preferably substitutions, insertions, or deletions) in comparison to any amino acid sequence of SEQ ID NO: 7, 15, 23, 31, 58, 62, 66, 70, 74; and/or
(f) LCDR3 of the light chain variable region is selected from any amino acid sequence of SEQ ID NO: 8, 16, 24, 32, 59, 63, 67, 71, 75, or a sequence having at least 80%, 85%, 90%, 91%, 92%, 93%, 94%, 95%, 96%, 97%, 98%, 99% or more identity to any amino acid sequence of SEQ ID NO: 8, 16, 24, 32, 59, 63, 67, 71, 75, or an amino acid sequence having one or more (preferably 2 or 3) conserved amino acid mutations (preferably substitutions, insertions, or deletions) in comparison to any amino acid sequence of SEQ ID NO: 8, 16, 24, 32, 59, 63, 67, 71, 75.

In alternative embodiments, HCDR1, HCDR2, HCDR3 of the heavy chain variable region, and LCDR1, LCDR2, LCDR3 of the light chain variable region are selected from the amino acid sequences of any of the following (1)-(14):
HCDR1 shown in SEQ ID NO: 2, HCDR2 shown in SEQ ID NO: 3, HCDR3 shown in SEQ ID NO: 4, LCDR1 shown in SEQ ID NO: 6, LCDR2 shown in SEQ ID NO: 7, and LCDR3 shown in SEQ ID NO: 8;
(2) HCDR1 shown in SEQ ID NO: 10, HCDR2 shown in SEQ ID NO: 11, HCDR3 shown in SEQ ID NO: 12, LCDR1 shown in SEQ ID NO: 14, LCDR2 shown in SEQ ID NO: 15, and LCDR3 shown in SEQ ID NO: 16;
(3) HCDR1 shown in SEQ ID NO: 18, HCDR2 shown in SEQ ID NO: 19, HCDR3 shown in SEQ ID NO: 20, LCDR1 shown in SEQ ID NO: 22, LCDR2 shown in SEQ ID NO: 23, and LCDR3 shown in SEQ ID NO: 24;
(4) HCDR1 shown in SEQ ID NO: 26, HCDR2 shown in SEQ ID NO: 27, HCDR3 shown in SEQ ID NO: 28, LCDR1 shown in SEQ ID NO: 30, LCDR2 shown in SEQ ID NO: 31, and LCDR3 shown in SEQ ID NO: 32;
(5) HCDR1 shown in SEQ ID NO: 41, HCDR2 shown in SEQ ID NO: 42, HCDR3 shown in SEQ ID NO: 43, LCDR1 shown in SEQ ID NO: 57, LCDR2 shown in SEQ ID NO: 58, and LCDR3 shown in SEQ ID NO: 59;
(6) HCDR1 shown in SEQ ID NO: 41, HCDR2 shown in SEQ ID NO: 42, HCDR3 shown in SEQ ID NO: 43, LCDR1 shown in SEQ ID NO: 61, LCDR2 shown in SEQ ID NO: 62, and LCDR3 shown in SEQ ID NO: 63;
(7) HCDR1 shown in SEQ ID NO: 41, HCDR2 shown in SEQ ID NO: 42, HCDR3 shown in SEQ ID NO: 43, LCDR1 shown in SEQ ID NO: 65, LCDR2 shown in SEQ ID NO: 66 and LCDR3 shown in SEQ ID NO: 67;
(8) HCDR1 shown in SEQ ID NO: 45, HCDR2 shown in SEQ ID NO: 46, HCDR3 shown in SEQ ID NO: 47, LCDR1 shown in SEQ ID NO: 57, LCDR2 shown in SEQ ID NO: 58, and LCDR3 shown in SEQ ID NO: 59;
(9) HCDR1 shown in SEQ ID NO: 45, HCDR2 shown in SEQ ID NO: 46, HCDR3 shown in SEQ ID NO: 47, LCDR1 shown in SEQ ID NO: 61, LCDR2 shown in SEQ ID NO: 62, and LCDR3 shown in SEQ ID NO: 63;
(10) HCDR1 shown in SEQ ID NO: 45, HCDR2 shown in SEQ ID NO: 46, HCDR3 shown in SEQ ID NO: 47, LCDR1 shown in SEQ ID NO: 65, LCDR2 shown in SEQ ID NO: 66, and LCDR3 shown in SEQ ID NO: 67;
(11) HCDR1 shown in SEQ ID NO: 45, HCDR2 shown in SEQ ID NO: 46, HCDR3 shown in SEQ ID NO: 47, LCDR1 shown in SEQ ID NO: 69, LCDR2 shown in SEQ ID NO: 70, and LCDR3 shown in SEQ ID NO: 71;
(12) HCDR1 shown in SEQ ID NO: 45, HCDR2 shown in SEQ ID NO: 46, HCDR3 shown in SEQ ID NO: 47, LCDR1 shown in SEQ ID NO: 73, LCDR2 shown in SEQ ID NO: 74, and LCDR3 shown in SEQ ID NO: 75;
(13) HCDR1 shown in SEQ ID NO: 49, HCDR2 shown in SEQ ID NO: 50, HCDR3 shown in SEQ ID NO: 51, LCDR1 shown in SEQ ID NO: 65, LCDR2 shown in SEQ ID NO: 66, and LCDR3 shown in SEQ ID NO: 67;
(14) HCDR1 shown in SEQ ID NO: 53, HCDR2 shown in SEQ ID NO: 54, HCDR3 shown in SEQ ID NO: 55, LCDR1 shown in SEQ ID NO: 65, LCDR2 shown in SEQ ID NO: 66, and LCDR3 shown in SEQ ID NO: 67.

In alternative embodiments, the anti-CCR8 antibody or antigen-binding fragment thereof comprises a heavy chain variable region and a light chain variable region, wherein,
the heavy chain variable region has an amino acid sequence of any one of the amino acids that are given in SEQ ID NO: 1, 9, 17, 25, 33, 34, 35, 36, 40, 44, 48, 52, or a sequence having at least 80%, 85%, 90%, 91%, 92%, 93%, 94%, 95%, 96%, 97%, 98%, 99% or more identity to the amino acid sequence that are given in SEQ ID NO: 1, 9, 17, 25, 33, 34, 35, 36, 40, 44, 48, 52 or an amino acid sequence having one or more (preferably 1, 2, 3, 4, 5, 6, 7, 8, 9, 10) conserved amino acid mutations (preferably substitutions, insertions, or deletions) in comparison to any of the amino acid sequences of SEQ ID NO: 1, 9, 17, 25, 33, 34, 35, 36, 40, 44, 48, 52;
(b) the light chain variable region has an amino acid sequence of any one of the amino acids that are given in SEQ ID NO: 5, 13, 21, 29, 37, 38, 39, 56, 60, 64, 68, 72, or a sequence having at least 80%, 85%, 90%, 91%, 92%, 93%, 94%, 95%, 96%, 97%, 98%, 99% or more identity to the amino acid sequence that are given in SEQ ID NO: 5, 13, 21, 29, 37, 38, 39, 56, 60, 64, 68, 72, or an amino acid sequence having one or more (preferably 1, 2, 3, 4, 5, 6, 7, 8, 9, 10) conserved amino acid mutations (preferably substitutions, insertions, or deletions) in comparison to any of the amino acid sequences of SEQ ID NO: 5, 13, 21, 29, 37, 38, 39, 56, 60, 64, 68, 72.

In alternative embodiments, the heavy chain variable region and the light chain variable region of the anti-CCR8 antibody or antigen-binding fragment thereof are selected from any of the amino acid sequences (1)-(26) below:
SEQ ID NO: 1 and SEQ ID NO: 5;
(2) SEQ ID NO: 9 and SEQ ID NO: 13;
(3) SEQ ID NO: 17 and SEQ ID NO: 21;
(4) SEQ ID NO: 25 and SEQ ID NO: 29;
(5) SEQ ID NO: 33 and SEQ ID NO: 37;
(6) SEQ ID NO: 33 and SEQ ID NO: 38;
(7) SEQ ID NO: 33 and SEQ ID NO: 39;
(8) SEQ ID NO: 34 and SEQ ID NO: 37;
(9) SEQ ID NO: 34 and SEQ ID NO: 38;
(10) SEQ ID NO: 34 and SEQ ID NO: 39;
(11) SEQ ID NO: 35 and SEQ ID NO: 37;
(12) SEQ ID NO: 35 and SEQ ID NO: 38;
(13) SEQ ID NO: 35 and SEQ ID NO: 39;
(14) SEQ ID NO: 36 and SEQ ID NO: 37;
(15) SEQ ID NO: 36 and SEQ ID NO: 38;
(16) SEQ ID NO: 36 and SEQ ID NO: 39;
(17) SEQ ID NO: 40 and SEQ ID NO: 56;
(18) SEQ ID NO: 40 and SEQ ID NO: 60;
(19) SEQ ID NO: 40 and SEQ ID NO: 64;
(20) SEQ ID NO: 44 and SEQ ID NO: 56;
(21) SEQ ID NO: 44 and SEQ ID NO: 60;
(22) SEQ ID NO: 44 and SEQ ID NO: 64;
(23) SEQ ID NO: 44 and SEQ ID NO: 68;
(24) SEQ ID NO: 44 and SEQ ID NO: 72;
(25) SEQ ID NO: 48 and SEQ ID NO: 64;
(26) SEQ ID NO: 52 and SEQ ID NO: 64.

In alternative embodiments, the antibody or antigen-binding fragment thereof is a mouse-derived antibody or antigen-binding fragment thereof, a chimeric antibody or antigen-binding fragment thereof, and/or a humanized antibody or antigen-binding fragment thereof.

In alternative embodiments, the anti-CCR8 antibody or antigen-binding fragment thereof also comprises an Fc region, and the Fc region is selected from mouse IgG1, IgG2a, IgG2b and/or IgG3, or from rat IgG1, IgG2a, IgG2b and/or IgG2c.

In alternative embodiments, the anti-CCR8 antibody or antigen-binding fragment thereof also comprises an Fc region, and the Fc region is selected from human IgG1, IgG2, IgG3 and/or IgG4 or from an amino acid sequence in the Fc region with one or more amino acid mutations (preferably substitutions, insertions, or deletions) with human IgG1, IgG2, IgG3, IgG4. In alternative embodiments, the Fc region can be modified, e.g., in the form of a Knob-in-Hole and the like.

The present invention also provides a nucleic acid molecule encoding the anti-CCR8 antibody or antigen-binding fragment thereof as described in any one of the above.

The present invention also provides a recombinant vector comprising the nucleic acid molecules described above.

The present invention also provides a recombinant cell comprising the above-described nucleic acid molecule and/or the above-described recombinant vector and capable of expressing said anti-CCR8 antibody or antigen-binding fragment thereof.

The present invention also provides a multifunctional fusion protein comprising the anti-CCR8 antibody or antigen-binding fragment thereof as described in any one of the above.

In alternative embodiments, the multifunctional fusion protein further comprises one or more second antibody or antigen-binding portions thereof that bind specifically to other antigens.

In alternative embodiments, the antigen binding the second antibody or antigen-binding portion thereof is selected from a tumor-associated antigen (TAA) or an immune checkpoint.

In alternative embodiments, the use of any of the above-described anti-CCR8 antibodies or antigen-binding fragments thereof, any of the above-described multifunctional fusion proteins in the preparation of a drug for the treatment and/or prevention and/or diagnosis of a disease.

In alternative embodiments, the use is achieved by one or more of tumor immunotherapy, cell therapy, and gene therapy.

In alternative embodiments, the use of any of the above-described anti-CCR8 antibodies or antigen-binding fragments thereof, any of the above-described multifunctional fusion proteins in the preparation of a drug for the treatment of cancer.

In alternative embodiments, the cancer is Breast cancer, ovarian cancer, lung cancer, liver cancer, melanoma, malignant glioma, head and neck cancer, colorectal cancer, stomach cancer, bladder cancer, pancreatic cancer, colon cancer, cervical cancer or related tumors.

The present invention also provides a pharmaceutical composition comprising the anti-CCR8 antibody or antigen-binding fragment thereof as described in any one of the above and an acceptable carrier, diluent or excipient.

The present invention also provides a pharmaceutical composition comprising any of the multifunctional fusion proteins described above and an acceptable carrier, diluent or excipient.

The anti-CCR8 antibody developed in the present invention can specifically recognize the human CCR8 protein, can block the signal pathway between CCR8 and CCL1/CCL18, can specifically recognize cells expressing CCR8, and has target specific ADCC activity on target cells expressing CCR8.

To help understand the invention set forth herein, the following explanation of abbreviations and definitions of terms are provided.

The following abbreviations are used in this invention:
CDR: Complementary determining regions in the variable region of the antibody;
HCDR: Complementary determining regions in the variable region of the antibody heavy chain;
LCDR: Complementary determining regions in the variable region of the antibody light chain;
FR: Antibody construct region, which is the amino acid residues other than CDR residues in the variable region of the antibody;
ELISA: Enzyme-Linked ImmunoSorbent Assay;
FACS: Fluorescence Activating Cell Sorter.

The term “antibody” refers to a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells, or from the immune serum of an animal, or from reconstruction by phage library screening. Alternatively, they may be partially or completely synthesized by using techniques such as genetic recombination. Preferred antibodies include, for example, antibodies to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Within these isotypes, the antibodies of the present invention may include IgG1, IgG2, IgG3, and/or IgG4.

The terms “antibody” and “immunoglobulin” are used interchangeably, and some of the antibodies used herein consist of an immunoglobulin molecule comprising two pairs of polypeptide chains, each pair having a light chain (LC) and a heavy chain (HC). Each heavy chain consists of a heavy chain variable region (VH) and a heavy chain constant region (CH). The heavy chain constant region consists of three structural domains (CH1, CH2 and CH3). Each light chain consists of a light chain variable region (VL) and a light chain constant region (CL), or only a light chain constant region (CL). The light chain constant region consists of a structural domain CL. The constant structural domain is not directly involved in antibody-antigen binding, but exhibits a variety of effector functions, such as the ability to mediate the binding of immunoglobulins to host tissues or factors, including various cells of the immune system (e.g., effector cells) and the first component of the classical complement system (C1q). The VH and VL regions can also be subdivided into regions with high variability (called complementary determining regions (CDRs)), with more conserved regions called framing regions (FRs) scattered in between. Each VH and VL consists of three CDRs and four FRs arranged from amino-terminal to carboxy-terminal in the following order: FR1, CDR1, FR2, CDR2, FR3, CDR3, FR4. The variable regions of each heavy chain/light chain pair (VH and VL) form the antigen-binding site, respectively.

The term “antigen-binding fragment” of an antibody refers to a polypeptide fragment of an antibody, such as a polypeptide fragment of a full-length antibody, which maintains the ability to specifically bind the same antigen bound by the full-length antibody and/or to compete with the full-length antibody for specific binding of the antigen, and which is also referred to as an “antigen-binding portion”. The antigen-binding portion of an antibody may be produced by recombinant DNA technology or by enzymatic or chemical breakage of the intact antibody. Non-limiting examples of antigen-binding fragments include Fab, Fab′, F(ab′)₂, Fd, Fv, dAb and complementary determining region (CDR) fragments, single chain antibodies (e.g., scFv), chimeric antibodies, diabodies, linear antibodies, nanobodies (e.g., technology from Ablynx), structural domain antibodies (e.g., technology from Domantis), and such peptides comprising at least a portion of an antibody sufficient to confer specific antigen-binding ability to the peptide.

The term “polypeptide” refers to a chain of amino acids of any length, irrespective of modification (e.g. phosphorylation or glycosylation). The term polypeptide includes proteins and fragments thereof. Polypeptides may be “exogenous”, meaning that they are “heterologous”, i.e., foreign to the host cell being utilized, e.g., human polypeptides produced by bacterial cells. Polypeptides are disclosed herein as sequences of amino acid residues. Those sequences are written from left to right in the direction from the amino terminus to the carboxy terminus. According to standard nomenclature, amino acid residue sequences are named in three-letter or one-letter codes as follows: Alanine (Ala, A), Arginine (Arg, R), Asparagine (Asn, N), Aspartic acid (Asp, D), Cysteine (Cys, C), Glutamine (Gln, Q), Glutamic acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y) and Valine (Val, V).

There are a variety of methods/systems in the field to define and characterize CDRs that have been developed and refined over the years, including Kabat, Chothia, IMGT, AbM, and Contact; Kabat is the most commonly used and defines CDRs based on sequence variability; Chothia defines CDRs based on sequence variability based on the position of the cyclic region of the structure; the IMGT system defines CDRs based on sequence variability and position within the variable domain structure; AbM is defined based on Oxford Molecules' AbM antibody modeling software and is a compromise between Kabat and Chothia; Contact defines CDRs based on the analysis of complex crystal structures, and is similar to Chothia in several respects.

Numbering of amino acid positions (e.g., amino acid residues in the Fc region) and target regions (e.g., CDR) in the anti-CCR8 antibodies of the present invention, using the Kabat system.

With respect to the percentage (%) amino acid sequence “identity” of the reference polypeptide sequence is defined as the percentage of amino acid residues in the candidate sequence that are identical to amino acid residues in the reference polypeptide sequence after comparing the sequences and introducing gaps where necessary to obtain the maximum percentage sequence identity. Comparisons for the purpose of determining percent amino acid sequence identity can be performed in a variety of ways within the skill of the art, such as using publicly available computer software, such as BLAST, BLAST-2, Clustal W, Megalign (DNASTAR) software, or the FASTA program package.

The term “monoclonal antibody” refers to a homogeneous antibody targeted only to a specific antigenic epitope. In contrast to common polyclonal antibody preparations that typically include different antibodies targeted to different clusters of antigenic determinants (epitopes), each monoclonal antibody is targeted to a single cluster of antigenic determinants on the antigen. The adjective “monoclonal” refers to the homogeneous characterization of an antibody and is not to be understood as requiring an antibody to be produced by any particular method. The monoclonal antibodies of the present invention are preferably produced by recombinant DNA methods or obtained by screening methods described elsewhere herein.

The term “mouse-derived antibody” is used herein to mean a monoclonal antibody prepared according to the knowledge and skill in the art. In some embodiments, the preparation involves injecting a test subject with an antigen and then isolating a hybridoma expressing an antibody having the desired sequence or functional properties. In some embodiments, the desired mouse-derived antibody is obtained by screening a mouse immune library.

The term “chimeric antibody” refers to an antibody that is formed by fusing a variable region of a mouse-derived antibody with a constant region of a human antibody, which can attenuate the immune response induced by the mouse-derived antibody. In some embodiments, to establish a chimeric antibody, a hybridoma secreting a mouse-derived specific monoclonal antibody is established, then the variable region gene is cloned from the mouse hybridoma cells, and then the constant region gene of the human antibody is cloned as needed, and the mouse variable region gene is inserted into a human vector after linking the mouse variable region gene and the human constant region gene to form a chimeric gene, and then finally, the chimeric antibody molecule is expressed in a eukaryotic industrial system or a prokaryotic industrial system.

The term “humanized antibody” means an antibody comprising at least one humanized antibody chain (i.e., at least one humanized light or heavy chain). The term “humanized antibody chain” (i.e., “humanized immunoglobulin chain”) refers to an antibody chain (i.e., light or heavy chain, respectively) having variable regions, the variable regions comprising substantial variable framework regions and complementarity determinations of human antibodies. Regions (CDRs) substantially derived from a non-human antibody (e.g., at least one CDR, two CDRs, or three CDRs). In some embodiments, the humanized antibody chain further comprises constant regions (e.g., in the case of a light chain, one constant region or a portion thereof, and in the case of a heavy chain, preferably three constant regions).

The term “host cell” refers to a cell that has been or is capable of being transformed with a nucleic acid sequence and thus expressing the selected target gene. The term includes the progeny of the parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is attached. The term includes vectors that are self-replicating nucleic acid structures and vectors that are incorporated into the genome of the host cell into which they are introduced. Certain vectors are capable of directing the expression of nucleic acids operably linked to them. Such vectors are referred to herein as “expression vectors”.

### BRIEF DESCRIPTION OF THE DRAWINGS

FIG. 1a shows the binding activity of mouse-derived anti-CCR8 antibody to CHOK1-CCR8 cells.

FIG. 1b shows the binding activity of mouse-derived anti-CCR8 antibody to CHOK1-CCR8 cells.

FIG. 2 shows the binding activity of chimeric anti-CCR8 antibody to CHOK1-CCR8 cells.

FIG. 3 shows the results of ADCC effect of chimeric anti-CCR8 antibody.

FIG. 4 shows the inhibition of β-arestin release from CCL1-activated Tango-H_CCR8-CHO-K1 cells by mouse-derived anti-CCR8 antibody.

FIG. 5 shows the in vivo efficacy data of the mouse-derived anti-CCR8 antibody.

FIG. 6a shows the binding activity of humanized anti-CCR8 antibody to CHOK1-CCR8 cells.

FIG. 6b shows the binding activity of humanized anti-CCR8 antibody to CHOK1-CCR8 cells.

FIG. 7a shows the results of ADCC effect of humanized anti-CCR8 antibody.

FIG. 7b shows the results of ADCC effect of humanized anti-CCR8 antibody.

FIG. 8 shows the inhibition of β-arestin release from CCL1-activated Tango-H_CCR8-CHO-K1 cells by humanized anti-CCR8 antibody.

### DETAILED DESCRIPTION

### Example 1 Animal Immunization

CCR8-CHO-K1 cells overexpressing human CCR8 were constructed by the lentiviral transduction of CHO cells method, and Balb/c mice were immunized with a total of 1×10⁷ cells inoculated with CCR8-284 cells as immunogen. A second immunization was performed half a month after the first immunization, followed by a next immunization at one-month intervals. Mouses were taken negative serum 3 days before immunization, and 50 μL of blood was taken by tail clipping 6 days after each immunization. Negative and immune sera were diluted proportionally (1:0.1K, 1:1K, 1:10K, 1:100K, 1:1000K, 1:10,000K), and serum titer was detected with CCR8-CHO-K1 cells overexpressing CCR8 by Cell-ELISA. When the titer result met the requirement and anti-human CCR8 antibody is detected at a dilution of >1:10K, mouse spleen and lymph nodes can be harvested.

### Example 2 Cell Fusion

B lymphocytes and lymph node cells for experiments were obtained from Balb/c mouse immunized four times with CCR8-293 cells, and the spleen and lymph nodes were placed in a cell sieve, which was then placed in a 50 mL centrifuge tube. DMEM was pipetted dropwise onto spleens for grinding to make a suspension of splenocytes, and centrifuged, at 1600 rpm, for 10 min, and supernatants were removed. B cells were resuspended with 2 mL of erythrocyte lysate, and lysed for 2 minutes at room temperature, then 30 mL of DMEM was added, and mixed and centrifuged at 1600 rpm for 10 minutes, and counted.

Myeloma cells SP2/0 (ATCC) were passaged one day before fusion so that the cells were in the logarithmic growth phase at the time of the experiment. Splenocytes and SP2/0 were mixed in a 2:1 ratio and centrifuged at 1600 rpm for 10 min. The mixed cells were washed twice with fusion solution and centrifuged at 1600 rpm for 10 minutes. Cells were suspended by adding fusion solution at a final cell density of 1×10⁷ cells/mL, and within 5 minutes, the cell suspension was transferred to the fusion chamber of an electrofusion apparatus (BTX; ECM 2001) for fusion. After completion of fusion the cells were moved from the fusion chamber to complete medium containing HAT and incubated at 37° C. for 60 min. After incubation the cells were spread in 96-well plates already containing feeder cells and incubated at 37° C. with 5% CO₂.

### Example 3 ELISA Method to Screen Positive Clones

Primary screening of the fusion supernatant was performed after 7 days of culture. The CCR8-CHO-K1 cells were spread into 96 enzyme plates (1.5×10⁴ cells/well), cultured for 36 h, manually washed twice with PBST, then fixed with 4% paraformaldehyde, and closed with 2% BSA, then poured off the closure solution and washed by plate washer for 3 times. The fusion supernatant was taken 100 μL/well and added into the closed enzyme labeling plate, warmed at 37° C. for 1 h and the liquid in the wells was discarded and washed by plate washer for 3 times. Sheep anti-mouse secondary antibody-HRP (Abcam; Ab6789) was diluted with 0.5% BSA, with 100 μL/well, at incubation at 37° C. The plate was washed with PBST in a plate washer for 6 times and patted dry on a plate paper, and Solarbio chromogenic solution (Solarbio; PR1200) was added to the plate wells with 100 μL/well and wrapped in aluminum foil, and the color development was performed at 37° C., with the light protected. The color development reaction was terminated by adding 1 mol/L HCl, and the data were read at 450 nm on an enzyme marker. The cell lines with OD450>1.0 in the test supernatant result were selected as the primary screening candidate positive cell lines, and the culture supernatant of the positive cell lines was aspirated and discarded, and new HAT complete medium was added.

### Example 4 FACS Method for Further Screening of Positive Clones

CCR8-CHO-K1 cells were taken and transferred to a centrifuge tube and centrifuged at 1000 rpm for 5 min. A 100 μL aliquot of 3×10⁵ stably expressing cells was divided into separate tubes and 100 μL of fusion supernatant was added. The cells were incubated at 4° C. for 60 min and then washed twice with excess FACS buffer. Cells were resuspended in 100 μL FACS buffer, and sheep anti-mouse secondary antibody-FITC (Abcam; ab6785) was added to the samples, then incubated for 30 minutes and washed twice with excess FACS buffer. Cells were fixated in fixation buffer and subsequently analyzed by flow cytometry. The FACS method was used to screen for antibodies that bind specifically to CCR8-CHO-K1 cells.

Two rounds of limited dilution were used for monocloning of hybridoma cells, which were detected by ELISA, and single clones with OD450>1.0 were selected as definitive candidate cell lines for passaging, and clones without monoclonal antibody were selected for the next subcloning with OD450>1.0.

### Example 5 Antibody Sample Production for Candidate Cell Lines

Hybridoma cells were cultured in T75 cell culture flasks until cell coverage was 80-90%. Discard the cell supernatant from 2 bottles, then 30 mL hybridoma-SFM was added and cultured at 37° C. with 5% CO₂. Cultivate for 2-3 days, the cell status and medium color were observed, if the medium color turned yellow, then 30 mL of new hybridoma-SFM could be added. It was cultured for 6-7 days, the culture supernatant was collected by low-speed centrifugation, and purification was carried out.

### Example 6 Binding Activity of Candidate Antibodies to Cells

The antibody to be tested was taken as the initial concentration of 50 μg/mL and diluted 5-fold with 6 gradients. The cells CCR8-CHO-K1 in the incubator were taken out, and the cell suspension was transferred to a 15 mL centrifuge tube, centrifuged, and counted by resuspension in PBS. A blank control group (Blank), a negative control group (NC), an experimental group, a positive control group (positive control BMK derived from the 1K17 sequence of US20210277129) and the irrelevant antibody group were kept. The cell suspension was spread in a 96-well plate according to about 3×10⁵ cells/well. Centrifugation (1000 rpm, 5 min) was performed, followed by washing with PBS, re-centrifugation, and repeated twice to remove media residues. The supernatant was discarded, 100 μL of primary antibody solution and irrelevant antibody solution were added to the experimental group and irrelevant antibody group, respectively, and the cells were resuspended and incubated at room temperature for 1 h. The blank group, and the negative control group were incubated with an equal amount of PBS.

After 1 h, PBS was added to wash twice. After the supernatant was discarded, 100 μL of fluorescent secondary antibody dilution (Mouse-derived secondary antibody from Abcam ab6785) was added to each sample group, except for the blank group which was added with 100 μL of PBS. After incubation at room temperature and protected from light for 0.5 h, centrifugation was performed and PBS added to wash the samples twice. After the supernatant was discarded, 120 μL of PBS was added to resuspend and sequentially flow cytometry was performed to detect the mean fluorescence intensity. The concentration of the antibody was logarithmically taken as the horizontal coordinate, and the Sigmoidaldose-response (VariableSlope) method (GraphPadPrism Software, GraphPadSoftware, San Diego, California) was chosen to perform a nonlinear regression to obtain the binding activity curves of the antibody on CCR8-CHO cells. The results are shown in FIGS. 1a and 1b.

As can be seen from FIG. 1a and FIG. 1b, the selected antibodies Ms-1, Ms-2, Ms-3, Ms-4, Ms-5, Ms-6, Ms-7, Ms-8, Ms-9, Ms-10, Ms-11, Ms-12, Ms-13, Ms-14, and Ms-15 all show good binding activity to CCR8-CHO, with binding activity superior to that of the positive control and much superior to the negative control.

### Example 7 Sequencing of Monoclonal Antibodies

The hybridomas with good binding activity to the engineered cell CCR8-CHO obtained by FACs assay screening were sequenced to obtain the heavy chain variable region and light chain variable region of Ms-1, Ms-2, Ms-8 and Ms-15, respectively.

(1) The amino acid sequences of HCDR1, HCDR2 and HCDR3 of the heavy chain variable region of Ms-1 are SEQ ID NO: 2, SEQ ID NO: 3 and SEQ ID NO: 4, respectively;

The amino acid sequence of the heavy chain variable region of Ms-1 is SEQ ID NO: 1;

The amino acid sequences of LCDR1, LCDR2 and LCDR3 of the light chain variable region of Ms-1 are SEQ ID NO: 6, SEQ ID NO: 7 and SEQ ID NO: 8, respectively;

The amino acid sequence of the light chain variable region of Ms-1 is SEQ ID NO: 5.

(2) The amino acid sequences of HCDR1, HCDR2 and HCDR3 of the heavy chain variable region of Ms-2 are SEQ ID NO: 10, SEQ ID NO: 11 and SEQ ID NO: 12, respectively;

The amino acid sequence of the heavy chain variable region of Ms-2 is SEQ ID NO: 9;

The amino acid sequences of LCDR1, LCDR2 and LCDR3 of the light chain variable region of Ms-2 are SEQ ID NO: 14, SEQ ID NO: 15 and SEQ ID NO: 16, respectively;

The amino acid sequence of the light chain variable region of Ms-2 is SEQ ID NO: 13.

(3) The amino acid sequences of HCDR1, HCDR2 and HCDR3 of the heavy chain variable region of Ms-8 are SEQ ID NO: 18, SEQ ID NO: 19 and SEQ ID NO: 20, respectively;

The amino acid sequence of the heavy chain variable region of Ms-8 is SEQ ID NO: 17;

The amino acid sequences of LCDR1, LCDR2 and LCDR3 of the light chain variable region of Ms-8 are SEQ ID NO: 22, SEQ ID NO: 23 and SEQ ID NO: 24, respectively;

The amino acid sequence of the light chain variable region of Ms-8 is SEQ ID NO: 21.

(4) The amino acid sequences of HCDR1, HCDR2 and HCDR3 of the heavy chain variable region of Ms-15 are SEQ ID NO: 26, SEQ ID NO: 27 and SEQ ID NO: 28, respectively;

The amino acid sequence of the heavy chain variable region of Ms-15 is SEQ ID NO: 25;

The amino acid sequences of LCDR1, LCDR2 and LCDR3 of the light chain variable region of Ms-15 are SEQ ID NO: 30, SEQ ID NO: 31 and SEQ ID NO: 32, respectively;

The amino acid sequence of the light chain variable region of Ms-15 is SEQ ID NO: 29.

### Example 8 Constructing and Expressing of Chimeric Antibodies

The antibody fragment obtained by sequencing in Example 7 was genetically synthesized and constructed into a human IgG framework. The antibody fragment was inserted into a PCDNA3.1 vector using molecular cloning technology, and constructed into a mammalian cell expression plasmid, which was introduced into the host cell line CHO cells by using liposomal transfection. The fermentation supernatant was obtained by using cell Fed-batch, and the supernatant of the fermentation broth was purified by affinity chromatography, and the constructed chimeric antibodies: CH-1, CH-2, CH-8 and CH-15 were finally purified.

The amino acid sequences of the CDR and variable structure domains of the chimeric antibodies CH-1, CH-2, CH-8, and CH-15 correspond to the amino acid sequences of the CDR and variable structure domains of Ms-1, Ms-2, Ms-8, and Ms-15 in Example 7, respectively.

The amino acid sequences of the heavy chain constant regions of the chimeric antibodies CH-1, CH-2, CH-8 and CH-15 are all identical, as shown in SEQ ID NO: 76, and the amino acid sequences of the light chain constant regions are shown in SEQ ID NO: 77.

### Example 9 Binding Activity of Chimeric Antibodies

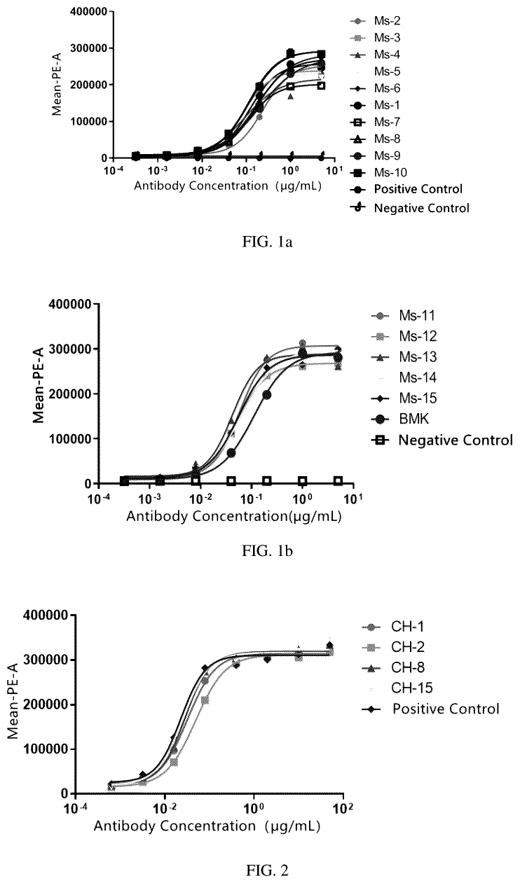

The binding activity of the chimeric antibodies was detected by FACs method, the secondary antibody was PE F(ab′) 2 sheep anti-human IgG Fc (Biolegend, Cat: 398004), and the assay procedure was the same as Example 6, and the results are shown in FIG. 2.

As can be seen from FIG. 2, the chimeric antibodies CH-1, CH-2, CH-8 and CH-15 all have very good binding activity with CCR8-CHO-K1 cells, comparable to the positive control activity.

### Example 10 ADCC Activity of Chimeric Antibodies to Target Cells

CCR8-CHOK1 cells overexpressing human CCR8 were used as target cells, centrifuged at 1000 rpm for 4 min at room temperature and resuspended using RPMI1640 basal medium (containing 5% FBS), and then spread in 96-well plates at 1×10⁴ cells/well and 50 μL/well. The antibody was diluted using RPMI1640 basal medium (containing 5% FBS) at a initial concentration of 10 μg/mL, while a 10-fold gradient of dilutions with 7 concentration gradients of 100 μL/well was followed. NK cells were resuspended and added to the corresponding wells at 50 μL/well, and E:T was 3:1 when the target cells were CCR8-CHOK1. target cell maximum lysis wells (M), target cell spontaneous release wells (ST), effector cell spontaneous release wells (SE), total volume-corrected blank wells (BV), and medium blank control wells (BM) were also set up. After standing for 10 min, it was centrifuged at 1000 rpm for 4 min at room temperature and incubated in a 5% CO₂, 37° C. CO₂ cell culture incubator for 4 h. Lysate was added to wells M and B-V 45 min ahead of time, mixed well, and centrifuged at 1000 rpm for 4 min at room temperature at the end of incubation. 50 μL of supernatant was aspirated to the LDH analysis plate, then 50 μL/well of substrate dissolved in analysis buffer (assay buffer) was added, and the reaction was carried out for 30 min at room temperature and protected from light, then 50 μL/well of termination solution was added, and it was left to stand for 10 min, and readings were taken at 490 nm to calculate the cell death rate.

$$Cell Death Rate ( \% ) = \frac{{ODvalue}_{sample well - ST - SE} \times 100}{{ODvalue}_{M - ST}}$$

The concentration of the constructed antibody was logarithmically taken as the horizontal coordinate, and the Sigmoidaldose-response (VariableSlope) method (GraphPadPrism Software, GraphPadSoftware, SanDiego, California) was chosen to perform a nonlinear regression to obtain the target antibody activity on the ADCC activity profile of the target cells.

As can be seen in FIG. 3, the IgG1 isotype control does not show the killing of CCR8-CHOK1 cells, while the chimeric antibodies CH-1, CH-2, CH-8, CH-15 and the positive control show the lytic death of CCR8-CHO-K1 cells in a concentration-dependent manner.

### Example 11 Antagonistic Activity of Antibodies Against CCL1

CCR8 is the only known receptor for CCL1, which binds to CCR8 on the cell surface and causes downstream signaling. Anti-CCR8 antibody can inhibit this signaling pathway by blocking CCR8, so the ability of anti-CCR8 antibody to block the CCL1-CCR8 pathway was tested using β-arrestin as a target, and Tango-H_CCR8-CHO-K1 (Jiman, GM-C13190) was used. Briefly, Switch-On Reagent (Jiman, cat: GM-040501A), an activator that induces CCR8 expression, was used to add to Tango-H_CCR8-CHO-K1 cells, and after 48 h of continuous culture, the cells were digested, and collected by centrifugation at 200 g. Cells were resuspended using the analytical buffer in ONE-Glo™ Luciferase Assay System (Biolegend), added to a 96-well assay plate according to the cell concentration of 1.5×10⁵ cells/mL in a volume of 100 μL, and incubated overnight in a cell culture incubator. The antibody was diluted using analytical buffer with a starting concentration of 15 μg/mL, while a 5-fold gradient dilution with 9 concentration gradients was performed, and 50 μL/well was incubated for 1 h. After that, 50 μL of CCL1 (Biolegend) was added as an activator with a final concentration of 0.06 μg/mL, and the fluorescence value was read by an enzyme marker after 7 h of incubation. The concentration of the constructed antibody was logarithmically taken as the horizontal coordinate, and the Sigmoidaldose-response (VariableSlope) method (GraphPadSoftware, SanDiego, California) was chosen for nonlinear regression to obtain the inhibition curve of the target antibody on CCL1 binding to CCR8, which triggers the downstream signaling molecule β-arrestin.

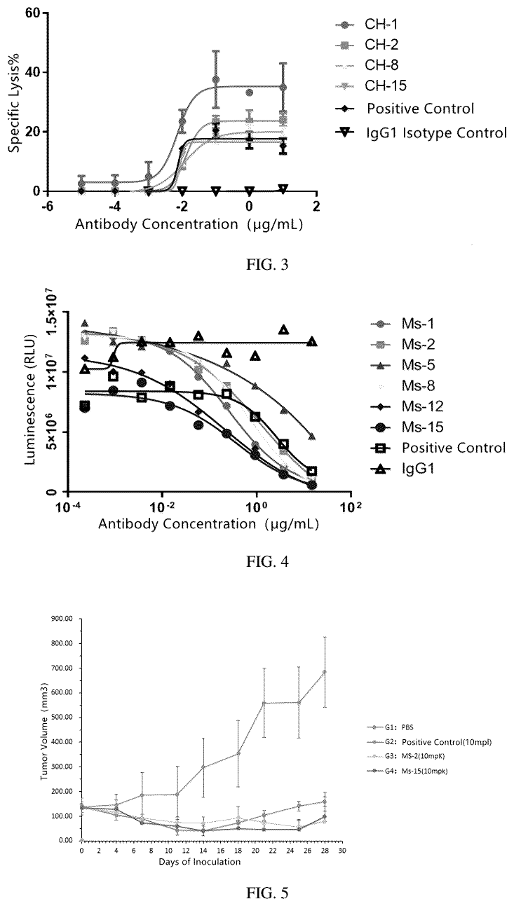

The results are shown in FIG. 4. IgG1, as the isotype control, could not inhibit the signaling value of β-arrestin activated by CCL1, and both the positive control and the candidate antibody show antagonistic activity with concentration-dependent activity.

### Example 12 In Vivo Drug Efficacy Assay of Mouse-Derived Antibodies

A C57 mouse (Biocytogen, C57BL/6-CCR8^{tm1(CCR8)}/Bcgen) subcutaneous tumor model of colon cancer MC38 cells was constructed with humanized CCR8, and in vivo antitumor efficacy of antibodies Ms-2 and Ms-15 was evaluated. MC38 cells were resuscitated, and cell culture was performed, then digested to make cell suspension. When the cells were cultured to the logarithmic growth phase, the cells were collected and the tumor cell suspension was injected subcutaneously into Balb/C nude mice, each inoculated with 100 μL of cell suspension containing 5×10⁵ cells. Tumor growth was observed, and when the subcutaneously grown tumors grew to about 100 mm³, the animals were randomly grouped according to tumor volume and given CCR8 antibody treatment. The experimental index was to examine whether the tumor growth was inhibited, retarded or cured. Tumor diameter was measured three times a week with vernier calipers. Tumor volume was calculated as V=0.5a×b2, with a and b denoting the long and short diameters of the tumor, respectively. Antibody and PBS were given intraperitoneally to grouped tumor-bearing mouse, respectively, and 10.0 mg/kg was administered twice a week for a total of 5 times.

The tumor suppression efficacy of the compounds was evaluated by TGI (%). Calculation of TGI (%): TGI (%)= [(1−(average tumor volume at the end of the administration of a treatment group-average tumor volume at the beginning of the administration of that treatment group))/(average tumor volume at the end of the treatment of the solvent control group−average tumor volume at the beginning of the treatment of the solvent control group)]×100%.

The tumor growth curves of mouse colon cancer MC38 cell subcutaneous tumor model of hormonal hCCR8 mouse given the antibody are shown in FIG. 5, where the horizontal coordinate indicates the number of days after the start of treatment and the vertical coordinate indicates the tumor volume. The candidate antibodies Ms-2 and Ms-15 have a high tumor inhibitory effect, which is significantly better than that of the PBS group and the positive control group, and the tumor suppression rate TGI (%) is greater than 50%.

### Example 13 Evaluation of Humanized Monoclonal Antibodies

The variable region of the chimeric antibody is subjected to humanization modification, with the design principle of not introducing protein modification sites such as glycosylation, deamidation, and isomerization, nor introducing integrin binding sites or cysteine. The reversion mutation of important amino acids in the framework region should maintain the original physicochemical and biochemical activity. The specific methods are as follows:

The mouse-derived sequences Ms-15 and Ms-2 were compared with human Germline sequences using the IgBLAST tool, respectively, and the FR was replaced with the human Germline sequence with the highest sequence similarity. The sequence of humanized antibody was obtained by selecting IGHV1 major class for Ms-2 heavy chain design template and IGKV3 major class for light chain design template, and IGHV3 major class for Ms-15 heavy chain design template and IGKV3 major class for light chain design template. And then on the basis of this humanization, several important amino acids affecting the affinity of the antibody were revertively mutated, i.e., mutated to the FR site of the original mouse origin. Percentage of humanization is the similarity ratio of the designed sequence Framework to the Germline sequence Framework, and the designed humanized sequences were compared with the human Germline sequences to select the sequences whose percentage of humanization of the antibody is 90% or more.

The heavy chain sequence of the chimeric antibody CH-2 was designed as four humanized sequences, Hu-2-H1, Hu-2-H2, Hu-2-H3 and Hu-2-H4, wherein the amino acid sequences of HCDR1, HCDR2 and HCDR3 are SEQ ID NO: 10, SEQ ID NO: 11 and SEQ ID NO: 12, respectively.

The light chain sequence of the chimeric antibody CH-2 was designed as three humanized sequences, Hu-2-L1, Hu-2-L2 and Hu-2-L3, wherein the amino acid sequences of LCDR1, LCDR2 and LCDR3 are SEQ ID NO: 14, SEQ ID NO: 15 and SEQ ID NO: 16, respectively.

The amino acid sequences of the variable regions of the four heavy chains and three light chains mentioned above are shown in Table 1, respectively.

TABLE 1

| | Amino Acid |
|---|---|
| Sequence | Sequences of |
| Name | Variable Regions |
| Hu-2-H1 | SEQ ID NO: 33 |
| Hu-2-H2 | SEQ ID NO: 34 |
| Hu-2-H3 | SEQ ID NO: 35 |
| Hu-2-H4 | SEQ ID NO: 36 |
| Hu-2-L1 | SEQ ID NO: 37 |
| Hu-2-L2 | SEQ ID NO: 38 |
| Hu-2-L3 | SEQ ID NO: 39 |

The above four humanized heavy chains and three humanized light chains were paired into 12 antibodies as shown in Table 2.

TABLE 2

| Sequence | Heavy Chain Variable | Light Chain Variable |
|---|---|---|
| Name | Region Sequence | Region Sequence |
| Hu-2-Z1 | Hu-2-H1 | Hu-2-L1 |
| Hu-2-Z2 | Hu-2-H1 | Hu-2-L2 |
| Hu-2-Z3 | Hu-2-H1 | Hu-2-L3 |
| Hu-2-Z4 | Hu-2-H2 | Hu-2-L1 |
| Hu-2-Z5 | Hu-2-H2 | Hu-2-L2 |
| Hu-2-Z6 | Hu-2-H2 | Hu-2-L3 |
| Hu-2-Z7 | Hu-2-H3 | Hu-2-L1 |
| Hu-2-Z8 | Hu-2-H3 | Hu-2-L2 |
| Hu-2-Z9 | Hu-2-H3 | Hu-2-L3 |
| Hu-2-Z10 | Hu-2-H4 | Hu-2-L1 |
| Hu-2-Z11 | Hu-2-H4 | Hu-2-L2 |
| Hu-2-Z12 | Hu-2-H4 | Hu-2-L3 |

The heavy chain of the chimeric antibody CH-15 was designed as a humanized sequence. CDR2 contains a DS (risk of aspartate isomerization), and the DS in the humanized sequence was mutated to ES, and another heavy chain was designed separately to revert the mutation. The heavy chain CDR3 also contains an RGD (integrin binding site), and was designed by mutating the RGD to RGE and RAD respectively on the basis of the revertant mutated sequence, adding two chains, thus totaling four chains, Hu-15-H1p1, Hu-15-H2p1, Hu-15-H2p1p2 and Hu-15-H2p1p3.

The CDR1 of the CH-15 light chain contains an NG (glycosylation risk), and was designed to mutate the NG to QG and NA, respectively, on the basis of the highest number of revertant mutations, and a total of five chains were designed for the light chain, which are Hu-15-L1, Hu-15-L2, Hu-15-L3, Hu-15-L3p1, and Hu-15-L3p2.

The amino acid sequences of the variable regions and CDRs of the above four heavy and five light chains, respectively, are shown in Table 3.

TABLE 3

| | Variable | | | |
|---|---|---|---|---|
| | Region | | | |
| Sequence | Sequence | CDR1 | CDR2 | CDR3 |
| Hu-15-H1p1 | SEQ ID | SEQ ID | SEQ ID | SEQ ID |
| | NO: 40 | NO: 41 | NO: 42 | NO: 43 |
| Hu-15-H2p1 | SEQ ID | SEQ ID | SEQ ID | SEQ ID |
| | NO: 44 | NO: 45 | NO: 46 | NO: 47 |
| Hu-15-H2p1p2 | SEQ ID | SEQ ID | SEQ ID | SEQ ID |
| | NO: 48 | NO: 49 | NO: 50 | NO: 51 |
| Hu-15-H2p1p3 | SEQ ID | SEQ ID | SEQ ID | SEQ ID |
| | NO: 52 | NO: 53 | NO: 54 | NO: 55 |
| Hu-15-L1 | SEQ ID | SEQ ID | SEQ ID | SEQ ID |
| | NO: 56 | NO: 57 | NO: 58 | NO: 59 |
| Hu-15-L2 | SEQ ID | SEQ ID | SEQ ID | SEQ ID |
| | NO: 60 | NO: 61 | NO: 62 | NO: 63 |
| Hu-15-L3 | SEQ ID | SEQ ID | SEQ ID | SEQ ID |
| | NO: 64 | NO: 65 | NO: 66 | NO: 67 |
| Hu-15-L3p1 | SEQ ID | SEQ ID | SEQ ID | SEQ ID |
| | NO: 68 | NO: 69 | NO: 70 | NO: 71 |
| Hu-15-L3p2 | SEQ ID | SEQ ID | SEQ ID | SEQ ID |
| | NO: 72 | NO: 73 | NO: 74 | NO: 75 |

The above 4 heavy chains and 5 light chains were paired to form 10 antibodies as shown in Table 4.

TABLE 4

| Antibody | Heavy Chain Variable | Light Chain Variable |
|---|---|---|
| Name | Region Sequence | Region Sequence |
| Hu-15-Z1 | Hu-15-H1p1 | Hu-15-L1 |
| Hu-15-Z2 | Hu-15-H1p1 | Hu-15-L2 |
| Hu-15-Z3 | Hu-15-H1p1 | Hu-15-L3 |
| Hu-15-Z4 | Hu-15-H2p1 | Hu-15-L1 |
| Hu-15-Z5 | Hu-15-H2p1 | Hu-15-L2 |
| Hu-15-Z6 | Hu-15-H2p1 | Hu-15-L3 |
| Hu-15-Z7 | Hu-15-H2p1 | Hu-15-L3p1 |
| Hu-15-Z8 | Hu-15-H2p1 | Hu-15-L3p2 |
| Hu-15-Z9 | Hu-15-H2p1p2 | Hu-15-L3 |
| Hu-15-Z10 | Hu-15-H2p1p3 | Hu-15-L3 |

The amino acid sequences in Table 3 were genetically synthesized and the above combinations of humanized sequences were expressed, and the humanized antibodies in Table 4 were prepared by constructing and expressing them according to the aforementioned method.

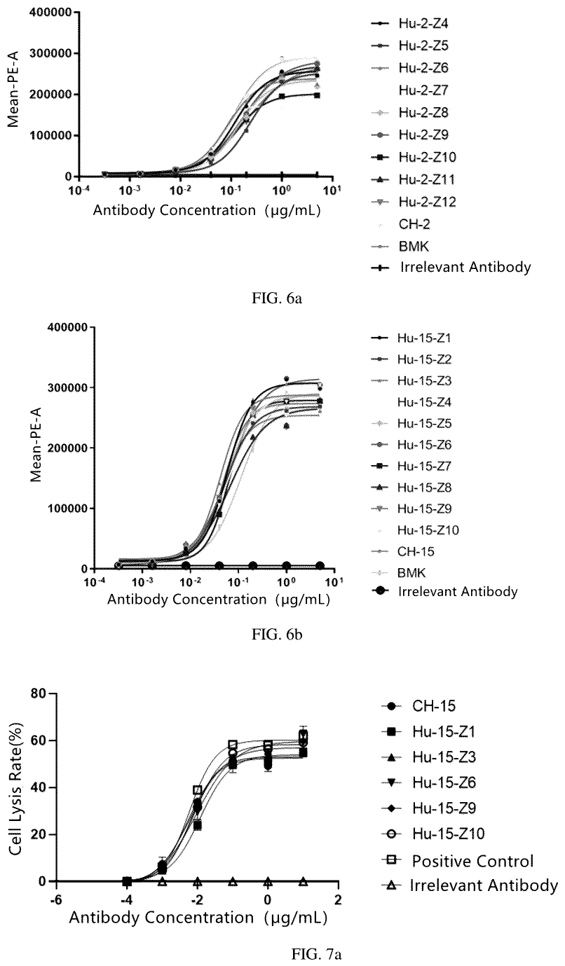

The binding activity of the humanized antibody was evaluated according to the steps of Example 6, and the results are shown in FIG. 6a and FIG. 6b. As can be seen from the results, both the humanized antibody and CHOK1-CCR8 cells show good binding activity, and the binding activity is comparable to that of the chimeric antibody and BMK (derived from the 1K17 sequence of US20210277129), and is much superior to that of the irrelevant antibody.

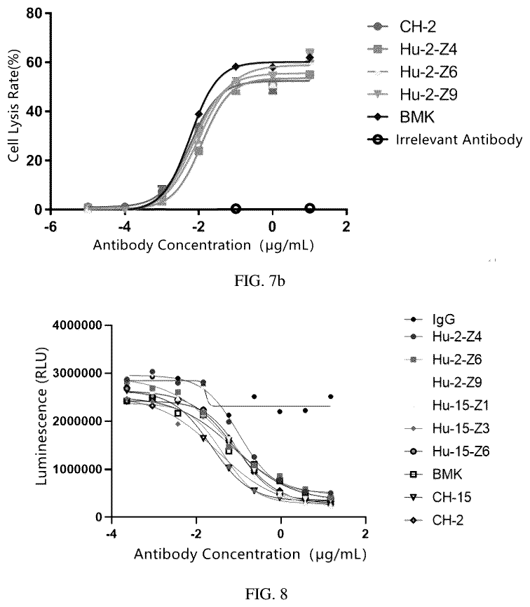

The ADCC activity of the humanized antibodies was evaluated according to the steps of Example 10, and the results are shown in FIG. 7a and FIG. 7b. As can be seen from the results, the humanized antibodies Hu-2-ZA, Hu-2-Z6, Hu-2-Z9, Hu-15-Z1, Hu-15-Z3 and Hu-15-Z6 all have good ADCC activity, and their cell lysis rate is comparable to that of the chimeric antibody and the BMK (derived from the 1K17 sequence of US20210277129) and is much superior to that of the irrelevant antibody.

The antagonist activity of humanized antibodies against the CCR-CCL1 signaling pathway was evaluated according to the steps of Example 11, and the results are shown in FIG. 8. The results indicated that both the positive control and the candidate antibody show antagonist activity with concentration-dependent activity, while the IgG isotype control is unable to inhibit the signaling value of CCL1 activation of β-arrestin.

1. An anti-CCR8 antibody or antigen-binding fragment thereof, comprising: a heavy chain variable region and a light chain variable region, wherein the heavy chain variable region comprises the heavy chain complementary determining regions HCDR1, HCDR2 and HCDR3, and the light chain variable region comprises the light chain complementary determining regions LCDR1, LCDR2 and LCDR3, wherein;
(a) HCDR1 of the heavy chain variable region is selected from any amino acid sequence of any of SEQ ID NO: 2, 10, 18, 26, 41, 45, 49, 53, or a sequence having at least 80%, 85%, 90%, 91%, 92%, 93%, 94%, 95%, 96%, 97%, 98%, 99% or more identity to the amino acid sequence of any of SEQ ID NO: 2, 10, 18, 26, 41, 45, 49, 53-, or an amino acid sequence having one or more conserved amino acid mutations compared to the amino acid sequence of any of SEQ ID NO: 2, 10, 18, 26, 41, 45, 49, 53;
(b) HCDR2 of the heavy chain variable region is selected from any amino acid sequence of any of SEQ ID NO: 3, 11, 19, 27, 42, 46, 50, 54, or a sequence having at least 80%, 85%, 90%, 91%, 92%, 93%, 94%, 95%, 96%, 97%, 98%, 99% or more identity to the amino acid sequence of any of SEQ ID NO: 3, 11, 19, 27, 42, 46, 50, 54, or an amino acid sequence having one or more conserved amino acid mutations as compared to the amino acid sequence of any of SEQ ID NO: 3, 11, 19, 27, 42, 46, 50, 54;
(c) HCDR3 of the heavy chain variable region is selected from any amino acid sequence of any of SEQ ID NO: 4, 12, 20, 28, 43, 47, 51, 55, or a sequence having at least 80%, 85%, 90%, 91%, 92%, 93%, 94%, 95%, 96%, 97%, 98%, 99% or more identity to the amino acid sequence of any of SEQ ID NO: 4, 12, 20, 28, 43, 47, 51, 55, or an amino acid sequence having one or more conserved amino acid mutations as compared to the amino acid sequence of any of SEQ ID NO: 4, 12, 20, 28, 43, 47, 51, 55;
(d) LCDR1 of the light chain variable region is selected from any amino acid sequence of SEQ ID NO: 6, 14, 22, 30, 57, 61, 65, 69, 73, or a sequence having at least 80%, 85%, 90%, 91%, 92%, 93%, 94%, 95%, 96%, 97%, 98%, 99% or more identity to any amino acid sequence of SEQ ID NO: 6, 14, 22, 30, 57, 61, 65, 69, 73, or an amino acid sequence having one or more conserved amino acid mutations in comparison to any amino acid sequence of SEQ ID NO: 6, 14, 22, 30, 57, 61, 65, 69, 73;
(e) LCDR2 of the light chain variable region is selected from any amino acid sequence of SEQ ID NO: 7, 15, 23, 31, 58, 62, 66, 70, 74, or a sequence having at least 80%, 85%, 90%, 91%, 92%, 93%, 94%, 95%, 96%, 97%, 98%, 99% or more identity to any amino acid sequence of SEQ ID NO: 7, 15, 23, 31, 58, 62, 66, 70, 74, or an amino acid sequence having one or more conserved amino acid mutations in comparison to any amino acid sequence of SEQ ID NO: 7, 15, 23, 31, 58, 62, 66, 70, 74; and/or
(f) LCDR3 of the light chain variable region is selected from any amino acid sequence of SEQ ID NO: 8, 16, 24, 32, 59, 63, 67, 71, 75, or a sequence having at least 80%, 85%, 90%, 91%, 92%, 93%, 94%, 95%, 96%, 97%, 98%, 99% or more identity to any amino acid sequence of SEQ ID NO: 8, 16, 24, 32, 59, 63, 67, 71, 75, or an amino acid sequence having one or more conserved amino acid mutations in comparison to any amino acid sequence of SEQ ID NO: 8, 16, 24, 32, 59, 63, 67, 71, 75.

2. The anti-CCR8 antibody or antigen-binding fragment thereof of claim 1, HCDR1, HCDR2, HCDR3 of the heavy chain variable region, and LCDR1, LCDR2, LCDR3 of the light chain variable region are selected from the amino acid sequences of any of the following (1)-(14):
(1) HCDR1 shown in SEQ ID NO: 2, HCDR2 shown in SEQ ID NO: 3, HCDR3 shown in SEQ ID NO: 4, LCDR1 shown in SEQ ID NO: 6, LCDR2 shown in SEQ ID NO: 7, and LCDR3 shown in SEQ ID NO: 8;
(2) HCDR1 shown in SEQ ID NO: 10, HCDR2 shown in SEQ ID NO: 11, HCDR3 shown in SEQ ID NO: 12, LCDR1 shown in SEQ ID NO: 14, LCDR2 shown in SEQ ID NO: 15, and LCDR3 shown in SEQ ID NO: 16;
(3) HCDR1 shown in SEQ ID NO: 18, HCDR2 shown in SEQ ID NO: 19, HCDR3 shown in SEQ ID NO: 20, LCDR1 shown in SEQ ID NO: 22, LCDR2 shown in SEQ ID NO: 23, and LCDR3 shown in SEQ ID NO: 24;
(4) HCDR1 shown in SEQ ID NO: 26, HCDR2 shown in SEQ ID NO: 27, HCDR3 shown in SEQ ID NO: 28, LCDR1 shown in SEQ ID NO: 30, LCDR2 shown in SEQ ID NO: 31, and LCDR3 shown in SEQ ID NO: 32;
(5) HCDR1 shown in SEQ ID NO: 41, HCDR2 shown in SEQ ID NO: 42, HCDR3 shown in SEQ ID NO: 43, LCDR1 shown in SEQ ID NO: 57, LCDR2 shown in SEQ ID NO: 58, and LCDR3 shown in SEQ ID NO: 59;
(6) HCDR1 shown in SEQ ID NO: 41, HCDR2 shown in SEQ ID NO: 42, HCDR3 shown in SEQ ID NO: 43, LCDR1 shown in SEQ ID NO: 61, LCDR2 shown in SEQ ID NO: 62, and LCDR3 shown in SEQ ID NO: 63;
(7) HCDR1 shown in SEQ ID NO: 41, HCDR2 shown in SEQ ID NO: 42, HCDR3 shown in SEQ ID NO: 43, LCDR1 shown in SEQ ID NO: 65, LCDR2 shown in SEQ ID NO: 66 and LCDR3 shown in SEQ ID NO: 67;
(8) HCDR1 shown in SEQ ID NO: 45, HCDR2 shown in SEQ ID NO: 46, HCDR3 shown in SEQ ID NO: 47, LCDR1 shown in SEQ ID NO: 57, LCDR2 shown in SEQ ID NO: 58, and LCDR3 shown in SEQ ID NO: 59;
(9) HCDR1 shown in SEQ ID NO: 45, HCDR2 shown in SEQ ID NO: 46, HCDR3 shown in SEQ ID NO: 47, LCDR1 shown in SEQ ID NO: 61, LCDR2 shown in SEQ ID NO: 62, and LCDR3 shown in SEQ ID NO: 63;
(10) HCDR1 shown in SEQ ID NO: 45, HCDR2 shown in SEQ ID NO: 46, HCDR3 shown in SEQ ID NO: 47, LCDR1 shown in SEQ ID NO: 65, LCDR2 shown in SEQ ID NO: 66, and LCDR3 shown in SEQ ID NO: 67;
(11) HCDR1 shown in SEQ ID NO: 45, HCDR2 shown in SEQ ID NO: 46, HCDR3 shown in SEQ ID NO: 47, LCDR1 shown in SEQ ID NO: 69, LCDR2 shown in SEQ ID NO: 70, and LCDR3 shown in SEQ ID NO: 71;
(12) HCDR1 shown in SEQ ID NO: 45, HCDR2 shown in SEQ ID NO: 46, HCDR3 shown in SEQ ID NO: 47, LCDR1 shown in SEQ ID NO: 73, LCDR2 shown in SEQ ID NO: 74, and LCDR3 shown in SEQ ID NO: 75;
(13) HCDR1 shown in SEQ ID NO: 49, HCDR2 shown in SEQ ID NO: 50, HCDR3 shown in SEQ ID NO: 51, LCDR1 shown in SEQ ID NO: 65, LCDR2 shown in SEQ ID NO: 66, and LCDR3 shown in SEQ ID NO: 67; and
(14) HCDR1 shown in SEQ ID NO: 53, HCDR2 shown in SEQ ID NO: 54, HCDR3 shown in SEQ ID NO: 55, LCDR1 shown in SEQ ID NO: 65, LCDR2 shown in SEQ ID NO: 66, and LCDR3 shown in SEQ ID NO: 67.

3. The anti-CCR8 antibody or antigen-binding fragment thereof of claim 1, further comprising a heavy chain variable region and a light chain variable region, wherein:
(a) the heavy chain variable region has an amino acid sequence of any one of the amino acids that are given in SEQ ID NO: 1, 9, 17, 25, 33, 34, 35, 36, 40, 44, 48, 52, or a sequence having at least 80%, 85%, 90%, 91%, 92%, 93%, 94%, 95%, 96%, 97%, 98%, 99% or more identity to the amino acid sequence that are given in SEQ ID NO: 1, 9, 17, 25, 33, 34, 35, 36, 40, 44, 48, 52 or an amino acid sequence having one or more conserved amino acid mutations in comparison to any of the amino acid sequences of SEQ ID NO: 1, 9, 17, 25, 33, 34, 35, 36, 40, 44, 48, 52; and
(b) the light chain variable region has an amino acid sequence of any one of the amino acids that are given in SEQ ID NO: 5, 13, 21, 29, 37, 38, 39, 56, 60, 64, 68, 72, or a sequence having at least 80%, 85%, 90%, 91%, 92%, 93%, 94%, 95%, 96%, 97%, 98%, 99% or more identity to the amino acid sequence that are given in SEQ ID NO: 5, 13, 21, 29, 37, 38, 39, 56, 60, 64, 68, 72, or an amino acid sequence having one or more conserved amino acid mutations in comparison to any of the amino acid sequences of SEQ ID NO: 5, 13, 21, 29, 37, 38, 39, 56, 60, 64, 68, 72.

4. The anti-CCR8 antibody or antigen-binding fragment thereof of claim 3, wherein the heavy chain variable region and the light chain variable region are selected from any of the following amino acid sequences (1)-(26):
(1) SEQ ID NO: 1 and SEQ ID NO: 5;
(2) SEQ ID NO: 9 and SEQ ID NO: 13;
(3) SEQ ID NO: 17 and SEQ ID NO: 21;
(4) SEQ ID NO: 25 and SEQ ID NO: 29;
(5) SEQ ID NO: 33 and SEQ ID NO: 37;
(6) SEQ ID NO: 33 and SEQ ID NO: 38;
(7) SEQ ID NO: 33 and SEQ ID NO: 39;
(8) SEQ ID NO: 34 and SEQ ID NO: 37;
(9) SEQ ID NO: 34 and SEQ ID NO: 38;
(10) SEQ ID NO: 34 and SEQ ID NO: 39;
(11) SEQ ID NO: 35 and SEQ ID NO: 37;
(12) SEQ ID NO: 35 and SEQ ID NO: 38;
(13) SEQ ID NO: 35 and SEQ ID NO: 39;
(14) SEQ ID NO: 36 and SEQ ID NO: 37;
(15) SEQ ID NO: 36 and SEQ ID NO: 38;
(16) SEQ ID NO: 36 and SEQ ID NO: 39;
(17) SEQ ID NO: 40 and SEQ ID NO: 56;
(18) SEQ ID NO: 40 and SEQ ID NO: 60;
(19) SEQ ID NO: 40 and SEQ ID NO: 64;
(20) SEQ ID NO: 44 and SEQ ID NO: 56;
(21) SEQ ID NO: 44 and SEQ ID NO: 60;
(22) SEQ ID NO: 44 and SEQ ID NO: 64;
(23) SEQ ID NO: 44 and SEQ ID NO: 68;
(24) SEQ ID NO: 44 and SEQ ID NO: 72;
(25) SEQ ID NO: 48 and SEQ ID NO: 64; and
(26) SEQ ID NO: 52 and SEQ ID NO: 64.

5. The anti-CCR8 antibody or antigen-binding fragment thereof claim 1, wherein the antibody or antigen-binding fragment thereof is a mouse-derived antibody or antigen-binding fragment thereof, a chimeric antibody or antigen-binding fragment thereof, and/or a humanized antibody or antigen-binding fragment thereof.

6. The anti-CCR8 antibody or antigen-binding fragment thereof claim 1, further comprising an Fc region, the Fc region is selected from mouse IgG1, IgG2a, IgG2b and/or IgG3, or from rat IgG1, IgG2a, IgG2b and/or IgG2c.

7. The anti-CCR8 antibody or antigen-binding fragment thereof of claim 1, further comprising an Fc region, the Fc region is selected from human IgG1, IgG2, IgG3 and/or IgG4 or an amino acid sequence in the Fc region with one or more amino acid mutations (preferably substitutions, insertions or deletions) with human IgG1, IgG2, IgG3, IgG4.

8. (canceled)

9. (canceled)

10. (canceled)

11. A multifunctional fusion protein, wherein the multifunctional fusion protein comprises the anti-CCR8 antibody or antigen-binding fragment thereof of claim 1.

12. The multifunctional fusion protein of claim 11, wherein the multifunctional fusion protein further comprises one or more second antibodies or antigen-binding portions thereof that bind specifically to other antigens.

13. The multifunctional fusion protein of claim 12, wherein the antigen binding the second antibody or antigen-binding portion thereof is selected from a tumor-associated antigen (TAA) or an immune checkpoint.

14. (canceled)

15. (canceled)

16. (canceled)

17. (canceled)

18. (canceled)

19. (canceled)
